(19) United States
(12) Patent Application Publication
(10) Pub. No.: US 2024/0307917 A1
(43) Pub. Date: Sep. 19, 2024

(54) Wireless Ultrasonic Transducer Apparatus

(71) Applicant: National Chin-Yi University of Technology, Taichung City (TW)

(72) Inventors: Jian-Xing Wu, Taichung City (TW); Hsiao-Chuan Liu, Cupertino, CA (US); Chih-Kuang Yeh, Taoyuan City (TW)

(21) Appl. No.: 18/184,104

(22) Filed: Mar. 15, 2023

(51) Int. Cl. B06B 1/06; A61B 8/00; G01N 29/24; H01M 50/102; H01Q 1/08; H05K 1/02

(52) U.S. Cl. CPC B06B 1/0607; A61B 8/4472; G01N 29/2481; H01M 50/102; H01Q 1/08; H05K 1/0213; B06B 2201/55; B06B 2201/76

(57) ABSTRACT

The invention relates to an wireless ultrasonic transducer apparatus comprises matching, wireless flexible antenna, antenna-based housing, Micro PCB board, and micro battery, wherein, micro battery is on the bottom base of the wireless ultrasonic transducer, then, micro PCB board is on micro battery, antenna-based housing is on micro PCB board, piezoelectric is on antenna-based housing, and then, matching is on wireless flexible antenna.

### BACKGROUND OF THE INVENTION

### 1. Field of the Invention

The invention relates to an ultrasonic transducer, particularly to a wireless ultrasonic transducer apparatus.

### 2. Description of the Prior Art

The ultrasonic transducer is one kind of electric device that generate or sense ultrasound energy, that is, the ultrasonic transducer is the electrical device that converts electromagnetic energy into mechanical energy (sound energy), usually made of piezoelectric ceramics or other magneto-strictive effect materials. General ultrasonic cleaners, ultrasonic atomizers, and B-ultrasound probes, abovementioned all are examples of the application of ultrasonic transducers.

Particularly, the ultrasonic transducer normally would be able to use for under water sonar and proximity measurement in air. According to Internet Web for Unictron Technologies Corporation of Taiwan, the Internet Web describes “structure and principle of ultrasonic transducer” as the following “the ultrasonic transducer uses the theory of time of flight”, the abovementioned “theory of time of flight” means the time difference between emitted ultrasound waves and reflected waves from the expected object, in order to calculate the distance between the ultrasonic transducer and the expected object by the abovementioned Internet Web. In addition, again, the abovementioned difference in the penetration energy of ultrasound waves between a single sheet and multiple sheets would be able to use for detection to determine whether expected objects overlap according to the abovementioned Internet Web of the Unictron Technologies Corporation of Taiwan. On the other hand, for ultrasonic detection, the expected object is to be normally detected by ultrasonic transducers regardless of solids, liquids, and powders.

FIG. 1 illustrates the schematic cross-section view for the conventional wired ultrasonic transducer. Therefore, in FIG. 1, the conventional ultrasonic transducer can be divided into some broad categories: connector 11, housing 12, backing 13, piezoelectric 14, matching 15, electric lead 16, and electric network 17. Wherein, connector 11 is an electric component inside the conventional ultrasonic transducer, and connector 11 is ready for connecting the outside electric wire in order to transit the signal out, and, piezoelectric 14 comprises piezoelectric ceramic disc transducer.

Nowadays even though the ultrasonic transducer is widely used in under water sonar, parking sensor, level sensor, double feed detection and flow meter, etc., however, unfortunately, inside the conventional ultrasonic transducer, still needs connector 11 being ready for connecting the outside electric wire in order to transit the signal out. That is, when the conventional wired ultrasonic transducer is elected to be used, the conventional wired ultrasonic transducer could be used by connecting with the electric wire, otherwise, the conventional wired ultrasonic transducer will be useless. Therefore, the electric wire will cause the most inconvenient and restriction for the application of the conventional wired ultrasonic transducer.

Therefore, how to solve the current shortcomings of conventional wired ultrasonic transducer will be the most urgent issue for the related people concerning. Indeed, the industry really should need a kind of the wireless ultrasonic transducer apparatus in the next quick coming generation.

### SUMMARY OF THE INVENTION

The invention relates to a capsule-type wireless ultrasonic transducer apparatus comprises matching, wireless flexible antenna, antenna-based housing, Micro PCB board, and micro battery, wherein, micro battery is on the bottom base of the wireless ultrasonic transducer, then, micro PCB board is on micro battery, sequently, antenna-based housing is on micro PCB board, capsule-type wireless flexible antenna is on antenna-based housing, and then, matching is on wireless flexible antenna.

A matching of the capsule-type wireless ultrasonic transducer apparatus can provide (have) a function for covering the top of the capsule-type wireless ultrasonic transducer.

A wireless flexible antenna of the capsule-type wireless ultrasonic transducer apparatus can provide (have) a function for generating or sensing ultrasound energy, and, converting electromagnetic energy into mechanical energy.

An antenna-based housing of the capsule-type wireless ultrasonic transducer apparatus can provide (have) a function for transmitting ultrasonic signals through the internal circuit

A micro PCB board of the capsule-type wireless ultrasonic transducer apparatus can provide (have) a function for converting the signal into a digital/analog adjustable signal, and then encrypt and send the digital/analog adjustable signal to the transmitting antenna.

A micro battery of the capsule-type wireless ultrasonic transducer apparatus can provide (have) a function for supporting an electric energy or electric power for the operation of the capsule-type wireless ultrasonic transducer.

The chip-type wireless ultrasonic transducer apparatus, which comprises antenna-based housing, flexible antenna, Micro PCB board, and micro battery. The antenna-based housing is sit on the chip, the flexible antenna is sit on the chip, the Micro PCB board is sit on the chip, and the micro battery is sit on the chip.

One of the advantages of the present invention, the goal of this wireless ultrasonic transducer apparatus is to develop the brand-new fabrication technology for Wi-Fi transmission ultrasonic transducer in order to achieve high-resolution and penetrating multi-level imaging technology through wireless transmission applications, and to solve the current shortcomings of conventional wired ultrasonic transducer.

Then, one of the advantages of the present invention, the use of this ultrasonic probe can effectively improve the inconvenience for connecting the conventional wired ultrasonic transducer.

One of the advantages of the present invention, the wireless ultrasonic transducer apparatus will achieve higher resolution under high excitation speed.

Furthermore, one of the advantages of the present invention, the wireless ultrasonic transducer apparatus will obtain high imaging speed and fusion images.

One of the advantages of the present invention, the wireless ultrasonic transducer apparatus can be practically applied to tissues and tumors, angiogenesis assessment and other important diagnostic indicators.

One of the advantages of the present invention, the wireless ultrasonic transducer apparatus will become the application potential of industrial/medical diagnostic analysis and evaluation systems.

As one of the advantages of the present invention, the wireless ultrasonic transducer apparatus uses the Micro PCB board to convert the signal into a digital/analog adjustable signal, and then encrypt and send the digital/analog adjustable signal to the transmitting antenna.

One of the advantages of the present invention, the probe housing is directly designed as a transmitting antenna, which can transmit ultrasonic signals through the internal circuit.

In addition, one of the advantages of the present invention, the wireless ultrasonic transducer apparatus is equipped with a replaceable Micro battery, providing 3 Volt, Capacity is 165 mAh, so that energy can maintain continuous wireless transmission for about 8 hours.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 illustrates the schematic cross-section view for the conventional wired ultrasonic transducer.

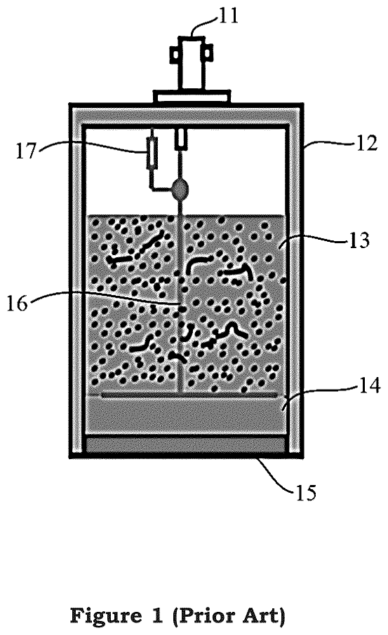

FIG. 2 illustrates the first embodiment for the schematic cross-section view of the wireless ultrasonic transducer according to the present invention.

FIG. 3 illustrates the second embodiment for the schematic cross-section view of the wireless ultrasonic transducer according to the present invention.

### DESCRIPTION OF THE PREFERRED EMBODIMENT

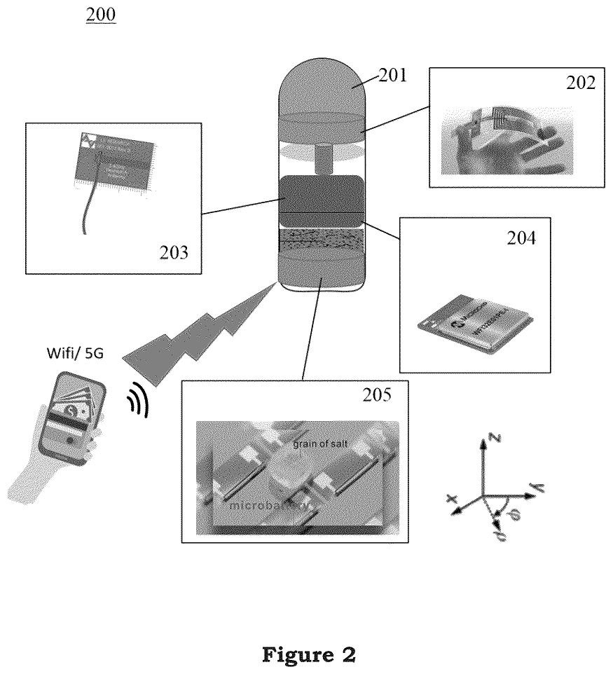

FIG. 2 illustrates the first embodiment for the schematic cross-section view of the wireless ultrasonic transducer apparatus 200 according to the present invention.

Thus, in FIG. 2, the wireless ultrasonic transducer apparatus 200, a kind of capsule-type wireless ultrasonic transducer 200, which comprises matching 201, wireless flexible antenna 202, antenna-based housing 203, Micro PCB board 204, and micro battery 205, wherein, micro battery 205 is on the bottom base of the wireless ultrasonic transducer apparatus 200, then, Micro PCB board 204 is on micro battery 205, and then, antenna-based housing 203 is on Micro PCB board 204, wireless flexible antenna 202 is on antenna-based housing 203, and then, matching 201 is on wireless flexible antenna 202.

Still as in FIG. 2, matching 201 of the first embodiment of the present invention for the cross-section for the capsule-type wireless ultrasonic transducer apparatus 200 can provide a function for covering the top of the capsule-type wireless ultrasonic transducer apparatus 200.

As FIG. 2 shown, wireless flexible antenna 202 of the second embodiment of the present invention for the cross-section for the capsule-type wireless ultrasonic transducer apparatus 200 can be used to transmit ultrasonic signals to the receiving end. Still as FIG. 2 shown, wireless flexible antenna 202 of the first embodiment of the present invention for the cross-section for the capsule-type wireless ultrasonic transducer apparatus 200 can provide a function for generating or sensing ultrasound energy, also, wireless flexible antenna 202 can be used to transmit ultrasonic signals to the receiving end. Wireless flexible antenna 202 is a kind of piezoelectric layer, which can convert electromagnetic energy into mechanical energy, therefore, wireless flexible antenna 202 could comprise piezoelectric ceramic disc transducer which is made of piezoelectric material polarized in the thickness direction. In fact, the above-mentioned piezoelectric materials include the PZT ceramic material, such as lead zirconate titanate, and the PMN-PT material, such as lead magnesium niobate-lead titanate materials, which are known for the high piezoelectric coefficient, high strain and low dielectric loss, extremely high electromechanical coupling coefficient.

Normally, there are two types of ultrasonic transducers as following:

### A. Magneto-Strictive Type Transducers

There are nickel sheet transducers and ferrite transducers that belong to magneto-striction. The electro-acoustic conversion efficiency of ferrite transducers is quite relatively low. In General, the efficiency drops after one or two years of use, and even almost loses the electro-acoustic conversion ability. The process of nickel sheet transducer is complicated and expensive, so that it is rarely used until so far.

### B. Piezoelectric Crystal Type Transducers

Wherein, the most mature and reliable device is the piezoelectric effect that realizes the mutual conversion of electrical energy and sound energy, which is called up as piezoelectric transducer. The electrical signal is converted up into mechanical vibration by the piezoelectric effect of the material. The transducer has high electro-acoustic conversion efficiency, cheap raw materials, convenient manufacture, and is not easy to be aged.

Sequently, as in FIG. 2, antenna-based housing 203 of the first embodiment of the present invention for the cross-section for the capsule-type wireless ultrasonic transducer apparatus 200 can provide a function for transmitting ultrasonic signals through the internal circuit. The probe housing is directly designed as a transmitting antenna, which can transmit ultrasonic signals through the internal circuit, the 2.4G module small antenna is used, and the ultrasonic transducer (probe) Housing is used as the built-in antenna to receive the reflected/scattered acoustic signals of the internal defects of the test piece at some different positions and angles. At this time, the frequency band of WIFI antenna copper tube antenna is 2400 MHz to 2500 MHz.

Consequesely, in FIG. 2, Micro PCB board 204 of the first embodiment of the present invention for the cross-section for the capsule-type wireless ultrasonic transducer apparatus 200 can provide a function for converting the signal into a digital/analog adjustable signal, and then encrypt and send the digital/analog adjustable signal to the transmitting antenna. Therefore, the present invention could use the Micro PCB board 204 to convert the signal into a digital/analog adjustable signal, and then encrypt and send the digital/analog adjustable signal to the transmitting antenna. Especially, such as “PIC32MZW1” features 1 MB embedded Flash Memory and 256 KB of SRAM, enabling embedded designers to rapidly build complex Internet of Things (IoT) software covering WLAN, TCP/IP stack, RTOS, cloud connectivity and applications. Many various types of peripherals such as Ethernet, USB, ADC, CVD touch buttons and CAN, which makes PIC32MZW1 as the perfect system core for most application functions.

Finally, as shown in FIG. 2, micro battery 205 of the first embodiment of the present invention for the cross-section for the capsule-type wireless ultrasonic transducer apparatus 200 can provide a function for supporting an electric energy or electric power for the operation of the capsule-type wireless ultrasonic transducer apparatus 200. The present invention is equipped with a replaceable Micro battery, providing 3 Volt, Capacity is 165 mAh, so that energy can maintain continuous wireless transmission for about 8 hours.

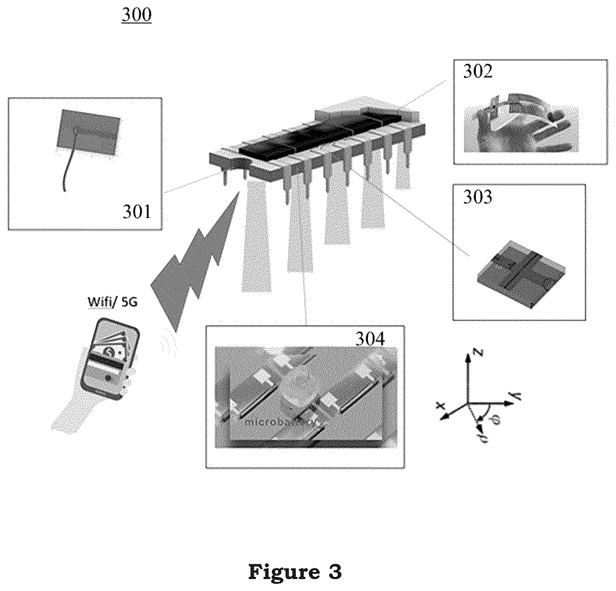

Furthermore, FIG. 3 illustrates the second embodiment for the schematic cross-section view of the wireless ultrasonic transducer apparatus 300 according to the present invention.

Thus, in FIG. 3, the wireless ultrasonic transducer apparatus 300, a kind of chip-type wireless ultrasonic transducer, which comprises antenna-based housing 301, flexible antenna 302, Micro PCB board 303, and micro battery 304 of the chip-type wireless ultrasonic transducer apparatus 300. The above-mentioned antenna-based housing 301 is installed on the chip, the flexible antenna 302 is installed on the chip, the antenna-based housing 303 is installed on the chip, and the micro battery 304 is installed on the chip.

FIG. 3 shows the appearance of this chip-type wireless ultrasonic transducer 300, which is made and completely packaged by a soft material around outside of the chip, and can be beneficial to the design for passing through the human body lumen, such as passing through the esophagus, large intestine, and vagina of the human. Especially, the abovementioned soft material is formed or provided by the polymer material, such as plastic materials.

As in FIG. 3, antenna-based housing 301 of the second embodiment of the present invention for the cross-section for the chip-type wireless ultrasonic transducer apparatus 300 can provide a function for transmitting ultrasonic signals through the internal circuit. The probe housing is directly designed as a transmitting antenna, which can transmit ultrasonic signals through the internal circuit, the 2.4G module small antenna is used, and the ultrasonic transducer (probe) Housing is used as the built-in antenna to receive the reflected/scattered acoustic signals of the internal defects of the test piece at some different positions and angles. At this time, the frequency band of WIFI antenna copper tube antenna is 2400 MHz to 2500 MHz.

As FIG. 3 shown, wireless flexible antenna 302 of the second embodiment of the present invention for the cross-section for the chip-type wireless ultrasonic transducer apparatus 300 can be used to transmit ultrasonic signals to the receiving end. Still as FIG. 3 shown, wireless flexible antenna 302 of the first embodiment of the present invention for the cross-section for the chip-type wireless ultrasonic transducer apparatus 300 can provide a function for generating or sensing ultrasound energy, also, wireless flexible antenna 302 can be used to transmit ultrasonic signals to the receiving end. Wireless flexible antenna 302 is a kind of piezoelectric layer, which can convert electromagnetic energy into mechanical energy, therefore, wireless flexible antenna 302 could comprise piezoelectric ceramic disc transducer which is made of piezoelectric material polarized in the thickness direction. In fact, the above-mentioned piezoelectric materials include the PZT ceramic material, such as lead zirconate titanate, and the PMN-PT material, such as lead magnesium niobate-lead titanate materials, which are known for the high piezoelectric coefficient, high strain and low dielectric loss, extremely high electromechanical coupling coefficient.

As FIG. 3 shown, Micro PCB board 303 of the first embodiment of the present invention for the cross-section for the chip-type wireless ultrasonic transducer apparatus 300 can provide a function for converting the signal into a digital/analog adjustable signal, inputting electric pulses, and then encrypt and send the digital/analog adjustable signal to the transmitting antenna. Therefore, the present invention could use the Micro PCB board 303 to convert the signal into a digital/analog adjustable signal, and then encrypt and send the digital/analog adjustable signal to the transmitting antenna. Especially, such as “PIC32MZW1” features 1 MB embedded Flash Memory and 256 KB of SRAM, enabling embedded designers to rapidly build complex Internet of Things (IOT) software covering WLAN, TCP/IP stack, RTOS, cloud connectivity and applications. Many various types of peripherals such as Ethernet, USB, ADC, CVD touch buttons and CAN, which makes PIC32MZW1 as the perfect system core for most application functions. On the other word, Micro PCB board 303 can firstly store the ultrasonic data in the chip memory, and then connect the data to the antenna through sorting and encryption methods to transmit the data to the receiving end.

Still as in FIG. 3, micro battery 304 of the first embodiment of the present invention for the cross-section for the chip-type wireless ultrasonic transducer apparatus 300 can provide a function for supporting an electric energy or electric power for the operation of the chip-type wireless ultrasonic transducer apparatus 300. The present invention is equipped with a replaceable Micro battery, providing 3 Volt, Capacity is 165 mAh, so that energy can maintain continuous wireless transmission for about 8 hours.

In addition, the medical ultrasonic transducer (such as ultrasonic probe) is an important part of the medical ultrasonic instrument system, the medical ultrasonic transducer occupies the very important position in the development of new medical instruments and medical research. In ultrasonic diagnosis, ultrasonic waves must be transmitted to the human body first, and then the reflected echoes of human tissue structure information must be received. The role of information conversion is the medical ultrasonic transducer, which completes an electro-acoustic and acoustic-electric conversion. The performance of the transducer is directly related to the performance of the medical ultrasonic equipment.

The principle of the wireless ultrasonic transducer apparatus is by analyzing the different characteristics of ultrasonic reflection rate and energy in materials with different densities, also, using the wireless antenna technology, the ultrasonic signal is transmitted to the receiving end. The goal of this invention is to develop the brand-new fabrication technology for Wi-Fi transmission ultrasonic transducer in order to achieve high-resolution and penetrating multi-level imaging technology through wireless transmission applications, and to solve the current shortcomings of conventional wired ultrasonic transducer.

The use of this ultrasonic probe can effectively improve the inconvenience for connecting the conventional wired ultrasonic transducer, achieve higher resolution under high excitation speed, obtain high imaging speed and fusion images, of course, the present invention can be practically applied to tissues and tumors, angiogenesis assessment and other important diagnostic indicators.

The present invention will become the application potential of industrial/medical diagnostic analysis and evaluation systems, the present invention use the Micro PCB board to convert the signal into a digital/analog adjustable signal, and then encrypt and send the digital/analog adjustable signal to the transmitting antenna, the probe housing is directly designed as a transmitting antenna, which can transmit ultrasonic signals through the internal circuit, the present invention is equipped with a replaceable micro battery, providing 3 Volt, Capacity is 165 mAh, so that energy can maintain continuous wireless transmission for about 8 hours.

It is understood that various other modifications will be apparent to and can be readily made by those skilled in the art without departing from the scope and spirit of the invention. Accordingly, it is not intended that the scope of the claims appended hereto be limited to the description as set forth herein, but rather that the claims be construed as encompassing all the features of patentable novelty that reside in the present invention, including all features that would be treated as equivalents thereof by those skilled in the art to which the invention pertains.

What is claimed is:

1. A wireless ultrasonic transducer, comprising:
a matching;
a wireless flexible antenna;
an antenna-based housing;
a micro PCB board; and
a micro battery, wherein said micro battery being on said bottom base of a wireless ultrasonic transducer, said micro PCB board being on micro battery, said antenna-based housing being on said micro PCB board, said wireless flexible antenna being on said antenna-based housing, and said matching is on said wireless flexible antenna.

2. The wireless ultrasonic transducer apparatus according to claim 1, wherein said matching comprises providing a function for covering a top of said wireless ultrasonic transducer.

3. The wireless ultrasonic transducer apparatus according to claim 1, wherein said wireless flexible antenna comprises providing a function for generating or sensing ultrasound energy, and converting electromagnetic energy into mechanical energy.

4. The wireless ultrasonic transducer apparatus according to claim 1, wherein said antenna-based housing comprises providing a function for transmitting ultrasonic signals through an internal circuit.

5. The wireless ultrasonic transducer apparatus according to claim 1, wherein an ultrasonic transducer housing comprises used as a built-in antenna.

6. The wireless ultrasonic transducer apparatus according to claim 1, wherein said micro PCB board comprises providing a function for converting the signal into a digital/analog adjustable signal, and then encrypt and send the digital/analog adjustable signal to the transmitting antenna.

7. The wireless ultrasonic transducer apparatus according to claim 1, wherein said micro battery comprises providing a function for supporting an electric energy or electric power for the operation of the wireless ultrasonic transducer.

8. A capsule-type wireless ultrasonic transducer apparatus having an antenna-based housing, comprising:
a matching;
a wireless flexible antenna;
an antenna-based housing;
a micro PCB board; and
a micro battery, wherein said micro battery being on said bottom base of a wireless ultrasonic transducer, said micro PCB board being on micro battery, said antenna-based housing being on said micro PCB board, said wireless flexible antenna being on said antenna-based housing, and said matching is on said wireless flexible antenna.

9. The capsule-type wireless ultrasonic transducer apparatus according to claim 8, wherein said matching comprises providing a function for covering a top of said wireless ultrasonic transducer.

10. The capsule-type wireless ultrasonic transducer apparatus according to claim 8, wherein said wireless flexible antenna comprises providing a function for generating or sensing ultrasound energy, and converting electromagnetic energy into mechanical energy.

11. The capsule-type wireless ultrasonic transducer apparatus according to claim 8, wherein said antenna-based housing comprises providing a function for transmitting ultrasonic signals through the internal circuit.

12. The capsule-type wireless ultrasonic transducer apparatus according to claim 8, wherein an ultrasonic transducer housing comprises used as a built-in antenna.

13. The capsule-type wireless ultrasonic transducer apparatus according to claim 8, wherein said micro PCB board comprises providing a function for converting the signal into a digital/analog adjustable signal, and then encrypt and send the digital/analog adjustable signal to the transmitting antenna.

14. The capsule-type wireless ultrasonic transducer apparatus according to claim 8, wherein said micro battery comprises providing a function for supporting an electric energy or electric power for the operation of the wireless ultrasonic transducer.

15. A chip-type wireless ultrasonic transducer apparatus having an antenna-based housing, comprising:
a wireless flexible antenna;
an antenna-based housing;
a micro PCB board; and
a micro battery, wherein said antenna-based housing being installed on a chip, said flexible antenna being installed on said chip, said antenna-based housing being installed on said chip, and said micro battery being installed on said chip.

16. The wireless ultrasonic transducer apparatus according to claim 15, wherein said wireless flexible antenna comprises providing a function for generating or sensing ultrasound energy, and converting electromagnetic energy into mechanical energy.

17. The wireless ultrasonic transducer apparatus according to claim 15, wherein said antenna-based housing comprises providing a function for transmitting ultrasonic signals through an internal circuit.

18. The wireless ultrasonic transducer apparatus according to claim 15, wherein an ultrasonic transducer housing comprises used as a built- in antenna.

19. The wireless ultrasonic transducer apparatus according to claim 15, wherein said micro PCB board comprises providing a function for converting the signal into a digital/analog adjustable signal, and then encrypt and send the digital/analog adjustable signal to the transmitting antenna.

20. The wireless ultrasonic transducer apparatus according to claim 15, wherein said micro battery comprises providing a function for supporting an electric energy or electric power for the operation of the wireless ultrasonic transducer.
